(19) United States
(12) Patent Application Publication
(10) Pub. No.: US 2011/0251413 A1
(43) Pub. Date: Oct. 13, 2011

(54) METHOD FOR PRODUCING PROPYLENE OXIDE

(75) Inventor: Fumikazu Yamashita, Osaka (JP)

(73) Assignee: Sumitomo Chemical Company, Limited

(21) Appl. No.: 13/140,869

(22) PCT Filed: Dec. 24, 2009
(86) PCT No.: PCT/JP2009/071855

§ 371 Date: Jun. 20, 2011

(30) Foreign Application Priority Data
Dec. 26, 2008 (JP) 2008-331845

(51) Int. Cl. C07D 301/04

(52) U.S. Cl. 549/533

(57) ABSTRACT

The present invention relates to a method for producing propylene oxide comprising the step of producing propylene oxide from hydrogen, oxygen and propylene in the presence of a noble metal catalyst, a titanosilicate and an organic sulfur compound in a mixed solvent of water and a nitrile compound, wherein the titanosilicate has a pore composed 12- or more-membered oxygen ring.

### TECHNICAL FIELD

The present invention relates to a method for producing propylene oxide from propylene, oxygen and hydrogen.

### BACKGROUND ART

As to the production of propylene oxide in which a titanosilicate is used as a catalyst and in which hydrogen, oxygen and propylene are used as raw materials, JP 2008-81488 A, JP 2008-106030 A and EP 1731515A1 respectively describe a method using a nitrile compound as a solvent.

### SUMMARY OF INVENTION

The present invention provides a novel method for producing propylene oxide.
Specifically, the present invention provides:
[1] a method for producing propylene oxide, comprising the step of producing propylene oxide from hydrogen, oxygen and propylene in the presence of a noble metal catalyst, a titanosilicate and an organic sulfur compound in a mixed solvent of water and a nitrile compound, wherein the titanosilicate has a pore composed 12- or more-membered oxygen ring;
[2] the method according to [1], wherein the organic sulfur compound is selected from the group consisting of a sulfide compound, a sulfoxide compound and a sulfone compound;
[3] the method according to [1], wherein the organic sulfur compound is selected from the group consisting of a sulfide compound and a sulfone compound;
[4] the method according to [2], wherein the sulfide compound is a dialkyl sulfide, an alkyl aryl sulfide or a diaryl sulfide;
[5] the method according to [2], wherein the sulfoxide compound is a dialkyl sulfoxide, an alkyl aryl sulfoxide or a diaryl sulfoxide;
[6] the method according to [2], wherein the sulfone compound is a dialkyl sulfone, an alkyl aryl sulfone or a diaryl sulfone;
[7] the method according to [1], wherein the organic sulfur compound is selected from the group consisting of a C2-C12 sulfide compound and a C2-C12 sulfone compound;
[8] the method according to [1], wherein the organic sulfur compound is dissolved in the mixed solvent;
[9] the method according to [1], wherein the nitrile compound is acetonitrile;
[10] the method according to [1], wherein the noble metal catalyst is a noble metal selected from the group consisting of palladium, platinum, ruthenium, rhodium, iridium, osmium and gold, or an alloy comprising two or more kinds of the noble metals;
[11] the method according to [10], wherein the noble metal catalyst is palladium, an alloy comprising palladium and the noble metal, or a mixture of the palladium and the alloy;
[12] the method according to [1], wherein the noble metal catalyst is supported on a carrier;
[13] the method according to [11], wherein the carrier is activated carbon;
[14] the method according to [1], wherein the organic sulfur compound and the noble metal catalyst are supported on a carrier;
[15] the method according to [1], wherein the titanosilicate is Ti-MWW, a Ti-MWW precursor or silylated Ti-MWW; and
[16] the method according to any one of [1] to [15], wherein anthraquinone is contained in the mixed solvent.

### EFFECT OF THE INVENTION

The present invention can improve the propylene oxide selectivity based on hydrogen and efficiently produce propylene oxide from propylene, oxygen and hydrogen.

### DESCRIPTION OF EMBODIMENTS

A method for producing propylene oxide according to the present invention comprises the step of synthesizing propylene oxide from hydrogen, oxygen and propylene in the presence of a noble metal catalyst, a titanosilicate having a pore, as described later, and an organic sulfur compound in a mixed solvent of water and a nitrile compound.

Examples of the organic sulfur compound include an organic sulfur compound selected from the group consisting of a sulfide compound, a sulfoxide compound and a sulfone compound. In the production method of the present invention, the organic sulfur compounds may be used alone or in combination of a plurality thereof.

The sulfide compound is generally represented by the following formula (1):

R¹—S—R² (1)
wherein R¹ and R² may be the same or different from each other and each independently represent an organic group, or may be bonded to each other to form a ring structure.

The sulfoxide compound is generally represented by the following formula (2):

R¹—S(O)—R² (2)
wherein R¹ and R² are as defined in the formula (1).

The sulfone compound is generally represented by the following formula (3):

R¹—S(O)₂—R² (3)
wherein R¹ and R² are as defined in the formula (1).

Examples of the organic group represented by R¹ or R² include an alkyl group which may be substituted, an aryl group which may be substituted or an alkenyl group which may be substituted.

Specific examples in which R¹ and R² are bonded together to form a part of a ring structure include an alkylene group, an alkenylene group and an arylene group. Examples of the alkylene group include a trimethylene group, a tetramethylene group and a pentamethylene group. Examples of the alkenylene group include a vinylene group, a propenylene group and a 2-butenylene group. Examples of the arylene group include a phenylene group, a naphthylene group and a biphenylene group.

Examples of the alkyl group which may be substituted include a C1-C20 linear or branched alkyl group such as a methyl group, an ethyl group, a propyl group, a butyl group, a pentyl group, a hexyl group, a heptyl group, an octyl group, a nonyl group, a decyl group, an undecyl group, a dodecyl group, a tridecyl group, a tetradecyl group, a pentadecyl group, a hexadecyl group, a heptadecyl group, an octadecyl group, a nonadecyl group and an icosyl group; and a C1-C20 alkyl group having a substituent selected from Group I as described later.

The aryl group which may be substituted may be a heteroaryl group containing a heteroatom such as nitrogen, oxygen or sulfur. Examples of the aryl group include a C4-C12 aryl group, specifically, a phenyl group, a biphenyl group, a 1-naphthyl group, a 2-naphthyl group, a furanyl group and a pyridyl group; and a C4-C12 aryl group having a substituent selected from Group I as described later.

Examples of the alkenyl group which may be substituted include a C2-C10 alkenyl group such as an ethenyl group, a 1-propenyl group, a 2-propenyl group, a 1-methylethenyl group, a 1-butenyl group, a 2-butenyl group, a 3-butenyl group, a 1-hexenyl group, a heptenyl group, an octenyl group, a nonenyl group and a decenyl group; and a C2-C10 alkenyl group having a substituent selected from Group I as described later.

Examples of the substituents selected from Group I include the following groups.

Group I: a halogen atom (a fluorine atom, a chlorine atom, a bromine atom or an iodine atom), a hydroxy group, a C1-C20 alkoxy group, a C6-C20 aryloxy group, an amino group, a mono(C1-C20 alkyl)amino group, a di(C1-C20 alkyl)amino group, a carboxy group, a C2-C21 alkoxycarbonyl group, a C7-C20 aryloxycarbonyl group, an alkanoyl group, an arylcarbonyl group, a formyl group, a C1-C20 alkylthio group, a C6-C20 arylthio group, a C1-C20 alkylsulfinyl group, a C6-C20 arylsulfinyl group, a C1-C20 alkylsulfonyl group and a C6-C20 arylsulfonyl group.

Examples of the sulfide compound preferably include a dialkyl sulfide, an alkyl aryl sulfide and a diaryl sulfide, the alkyl or aryl group of which may have a substituent selected from the Group I, more preferably include a (C1-C20 alkyl)sulfide, a (C1-C20 alkyl)(C6-C20 aryl)sulfide and a di(C6-C20 aryl)sulfide, the alkyl or aryl group of which may have a substituent selected from the Group I, and still more preferably include a di(C1-C5 alkyl)sulfide, a (C1-C5 alkyl)(C6-C10 aryl)sulfide and a di(C6-C10 aryl) sulfide, the alkyl or aryl group of which may have a hydroxyl group.

Specific examples of the sulfide compound include dimethyl sulfide, diethyl sulfide, dipropyl sulfide, isopropyl methyl sulfide, diisopropyl sulfide, dibutyl sulfide, t-butyl methyl sulfide, di-t-butyl sulfide, bis(methylthio)methane, thiodiglycol, 2-(ethylthio)ethanol, 2-(isopropylthio)ethanol, 2,2′-thiodiethanol, 3,6-dithia-1,8-octanediol, thiomorpholine, ethyl vinyl sulfide, tetrahydrothiophene, diphenylsulfide, methyl phenyl sulfide, 4-methoxythioanisole, 2-(phenylthio)ethanol, methoxymethyl phenyl sulfide, bis(4-hydroxyphenyl)sulfide, bis(4-aminophenyl)sulfide, bis(2-aminophenyl)sulfide, bis(phenylthio)methane, thioxanthene-9-one, 2-chlorothioxanthone, thianthrene, 2-aminophenyl phenyl sulfide, 4,4′-dipyridyl sulfide, 1,2-bis(phenylthio)ethane, phenyl trifluoromethyl sulfide, phenylvinylsulfide, allyl phenyl sulfide, 2-(methylthio)aniline, 2-(methylthio)pyridine, 2-fluorothioanisole, 2-chlorothioanisole, 2-bromothioanisole, 4-bromothioanisole, 4-(methylthio)benzaldehyde, (phenylthio)acetonitrile, 2-methoxythioanisole, 2-methyl-3-(methylthio)furan and S-phenyl thioacetate.

In the present invention, examples of the sulfide compound preferably include dibutyl sulfide, 2,2′-thiodiethanol, methyl phenyl sulfide and diphenyl sulfide, and more preferably include methyl phenyl sulfide and diphenyl sulfide.

Examples of the sulfoxide compound preferably include a dialkyl sulfoxide, an alkyl aryl sulfoxide and a diaryl sulfoxide, the alkyl or aryl group of which may have a substituent selected from the Group I, more preferably include a (C1-C20 alkyl)sulfoxide, a (C1-C20 alkyl)(C6-C20 aryl)sulfoxide and a di(C6-C20 aryl)sulfoxide, and still more preferably include a (C1-C5 alkyl)sulfoxide, a (C1-C5 alkyl)(C6-C10 aryl)sulfoxide and a di(C6-C10 aryl)sulfoxide.

Specific examples of the sulfoxide compound include dimethyl sulfoxide, diethyl sulfoxide, dipropyl sulfoxide, dibutyl sulfoxide, tetramethylene sulfoxide, diphenyl sulfoxide, methyl phenyl sulfoxide, phenyl vinyl sulfoxide, dibenzyl sulfoxide, methyl (methylsulfinyl)methyl sulfide and 1,2-bis(phenylsulfinyl)ethane.

Examples of the sulfone compound preferably include a dialkyl sulfone, an alkyl aryl sulfone, a diaryl sulfone, a cyclic sulfone having an alkylene structure and a sulfone compound in which the alkyl or aryl group has a substituent selected from the Group I, more preferably include a di(C1-C20 alkyl)sulfone, a (C1-C20 alkyl)(C6-C20 aryl)sulfone, a di(C6-C20 aryl)sulfone and a cyclic sulfone having a C2-C8 alkylene structure, and still more preferably include a di(C1-C5 alkyl)sulfone, a (C1-C5 alkyl)(C6-C10 aryl)sulfone and a di(C6-C10 aryl) sulfone and a cyclic sulfone having a C2-C6 alkylene structure.

Specific examples of the sulfone compound include dimethyl sulfone, ethyl methyl sulfone, isopropyl methylsulfone, dipropyl sulfone, dibutylsulfone, 2-(hydroxymethyl) ethyl sulfone, 3-sulfolene, divinyl sulfone, sulfolane, methyl phenyl sulfone, ethyl phenyl sulfone, phenyl vinyl sulfone, diphenyl sulfone, bis(vinylsulfonyl)methane, 1,4-thioxane-1,1-dioxide, 3 -methylsulfolane, (methylsulfonyl)acetonitrile, 4-chlorophenyl methyl sulfone, ethyl(phenylsulfonyl)acetate and allyl phenyl sulfone. In the present invention, examples of the sulfone compound preferably include dimethyl sulfone, diphenyl sulfone and sulfolane, and more preferably include diphenyl sulfone.

In the present invention, the method for supplying the organic sulfur compound is not particularly limited, and for example, the sulfur compound may be used by dissolving in a solvent, or may be used by supporting on a noble metal catalyst. As the sulfur compound, such a compound may be used that is converted into the sulfide compound, sulfoxide compound or sulfone compound by the oxidation with oxygen or reduction with hydrogen in a reactor.

The upper limit of the amount of the organic sulfur compound to be used is generally an effective amount of the compound, specifically an amount such that the amount of the product propylene oxide per noble metal catalyst is not decreased compared to the case the organic sulfur compound is not presented. The amount used is preferably 0.1 μmol/kg to 500 mmol/kg, more preferably 1 μmol/kg to 50 mmol/kg and still more preferably 1 μmol/kg to 5 mmol/kg, per kg of the mixed solvent.

Examples of the noble metal catalyst in the present invention include a noble metal such as palladium, platinum, ruthenium, rhodium, iridium, osmium, or alloys thereof. Examples of a preferred noble metal include palladium, platinum and gold. An example of a more preferred noble metal is palladium. As palladium, for example, a palladium colloid may be used (for example, refer to Example 1 in JP 2002-294301 A).

As the noble metal catalysts, the noble metals or alloys may be used alone or in combination of two or more kinds thereof. For example, in the case of using palladium as the noble metal catalyst, a metal, such as platinum, gold, rhodium, iridium and osmium, except for palladium, may be used by mixing with palladium. Examples of a preferred metal except for palladium include gold and platinum.

The noble metals are preferably used in the form of being supported on a carrier.

The noble metals may be used by being supported on a titanosilicate as described later. The noble metals may be used by being supported on oxides such as silica, alumina, titania, zirconia and niobia, hydrates such as niobic acid, zirconic acid, tungstic acid and titanic acid, carbon and a mixture thereof. Among the carriers other than a titanosilicate, examples of a preferred carrier include carbon. Examples of known carbon carriers include activated carbon, carbon black, graphite and carbon nanotube. Examples of a more preferred carbon carrier include activated carbon.

Examples of the methods for supporting a noble metal on a carrier include the method which comprises impregnating the carrier in the solution containing a noble metal compound, and then reducing the compound on the carrier; the method which comprises impregnating the carrier in the solution containing a colloid of noble metal, and then calcining the colloid on the carrier.

Examples of the noble metal compound include a palladium compound. Examples of the palladium compound include tetravalent palladium compounds such as sodium hexachloropalladium (IV) tetrahydrate and potassium hexachloropalladium (IV); and divalent palladium compounds such as palladium chloride (II), palladium bromide (II), palladium acetate (II), palladium acetylacetate (II), dichlorobis(benzonitrile) palladium (II), dichlorobis(acetonitrile)palladium (II), dichloro(bis(diphenylphosphino)ethane)palladium (II), dichlorobis(triphenylphosphine) palladium (II), dichlorotetraammine palladium (II), dibromotetraammine palladium (II), dichloro(cycloocta-1,5-diene) palladium (II) and palladium trifluoroacetate (II).

Examples of a method for converting a noble metal compound into a noble metal catalyst include reduction.

A commercially available colloidal solution of a noble metal may be used. Alternatively, for example, the colloidal solution can be prepared by dispersing noble metal particles with a dispersant such as citric acid, polyvinylalcohol, polyvinylpyrrolidone and sodium hexametaphosphate.

Examples of a method for reducing the noble metal compound supported on a carrier include a method of reducing the noble metal compound with a reducing agent in a liquid or gas phase. Examples of a reducing agent for the reduction in a gas phase include hydrogen. The temperature suitable for the reduction varies depending on the kind of noble metal compound supported on a carrier, but is usually 0° C. to 500° C. In addition, the noble metal compound may be reduced with an ammonia gas generated during thermal decomposition in an inert gas atmosphere. Although the reduction temperature varies depending on the kind of the noble metal compound or the like, in the case of using dichlorotetraammine palladium (II) as the noble metal compound, the reduction temperature is in the range of preferably 100 to 500° C. and more preferably 200 to 350° C. Examples of the reducing agent for the reduction in a liquid phase include hydrogen, hydrazine monohydrate, formaldehyde and sodium borohydride. In the case of using hydrazine monohydrate and formaldehyde, the reduction may be carried out by the addition of an alkali.

The catalyst with the noble metal being supported on the carrier contains the metal in an amount of usually 0.01 to 20% by weight and preferably 0.1 to 10% by weight.

In the present invention, the amount of the noble metal catalyst is preferably 0.00001 to 1% by mass and more preferably 0.0001 to 0.1% by mass, based on the mixed solvent.

When the noble metal catalyst is supported on a carrier, the organic sulfur compound may be supported on the carrier. The catalyst on which the organic sulfur compound is supported can be prepared, for example, by a method described in Advanced Synthesis and Catalysis 350, 406-410, (2008), specifically which comprises stirring the carrier supporting the noble metal catalyst and the organic sulfur compound in an alcohol and then washing the resulting mixture with an alcohol and an organic solvent. In the catalyst on which the organic sulfur compound and the noble metal are supported, the amount of the organic sulfur compound is in the range of usually 0.01 to 25% by mass and preferably 0.05 to 15% by mass, in terms of sulfur atom.

In the present invention, a titanosilicate acts as a catalyst for the reaction of producing propylene oxide from propylene.

A titanosilicate is a generic name for a silicate having a four-coordinate Ti (titanium atom), which has a porous structure. In the present invention, a titanosilicate means a titanosilicate having substantially 4-coordination Ti, which shows the maximum absorption peak in the wavelength range of 210 nm to 230 nm in the ultraviolet visible absorption spectra in the wavelength range of 200 nm to 400 nm (for example, refer to FIGS. 2 (d) and (e) in Chemical Communications 1026-1027, (2002)). The ultraviolet visible absorption spectra can be measured by a diffuse reflectance method using an ultraviolet-visible spectrophotometer equipped with a diffuse reflection apparatus.

The titanosilicate in the present invention has a pore composed 12- or more-membered oxygen ring.

In the present description, a pore means a pore composed of a Si—O bond or a Ti—O bond. The pore may be a hemispherical pore, which is referred to as a side pocket. In other words, the pore is not required to penetrate primary particles of the titanosilicate.

The “12- or more-membered oxygen ring” means a ring structure which has 12 or more oxygen atoms at (a) the cross-section of the narrowest portion of a pore or (b) the pore opening.

The fact that the titanosilicate has a pore composed 12- or more-membered oxygen ring is generally confirmed by X-ray diffraction pattern analysis, but can be conveniently confirmed by comparing the X-ray diffraction pattern if the structure is known.

Examples of the titanosilicate in the present invention include the titanosilicates described in the following 1 to 5.

### 1. Crystalline Titanosilicates Having a Pore Composed 12-Membered Oxygen Ring:

According to the structure code terminology used by the International Zeolite Association (IZA), Ti-Beta having a BEA structure (for example, one described in the Journal of Catalysis 199, 41-47, (2001)), Ti-ZSM-12 having an MTW structure (for example, one described in Zeolites 15, 236-242, (1995)), Ti-MOR having an MOR structure (for example, one described in The Journal of Physical Chemistry B 102, 9297-9303, (1998)), Ti-ITQ-7 having an ISV structure (for example, one described in Chemical Communications 761-762, (2000)), Ti-MCM-68 having an MSE structure (for example, one described in Chemical Communications 6224-6226, (2008)), Ti-MWW having an MWW structure (for example, one described in Chemistry Letters 774-775, (2000)), and the like.

### 2. Crystalline Titanosilicates Having a Pore Composed 14-Membered Oxygen Ring:

Ti-UTD-1 having a DON structure (for example, one described in Studies in Surface Science and Catalysis 15, 519-525, (1995)), and the like.

### 3. Lamellar Titanosilicates Having a Pore Composed 12-Membered Oxygen Ring:

A Ti-MWW precursor (for example, one described in EP 1731515A1), Ti-YNU-1 (for example, one described in Angewandte Chemie International Edition 43, 236-240, (2004)), Ti-MCM-36 (for example, one described in Catalysis Letters 113, 160-164, (2007)), Ti-MCM-56 (for example, one described in Microporous and Mesoporous Materials 113, 435-444, (2008)), and the like.

### 4. Mesoporous Titanosilicates:

Ti-MCM-41 (for example, one described in Microporous Materials 10, 259-271, (1997)), Ti-MCM-48 (for example, one described in Chemical Communications 145-146, (1996)), Ti-SBA-15 (for example, one described in Chemistry of Materials 14, 1657-1664, (2002)), and the like.

### 5. Silylated Titanosilicates:

Compounds obtained by silylating the titanosilicates described in the above 1 to 4 such as silylated Ti-MWW.

The “12-membered oxygen ring” means a ring structure which has 12 oxygen atoms at (a) the cross-section of the narrowest portion of a pore or (b) the pore opening. The “14-membered oxygen ring” means a ring structure which has 14 oxygen atoms at the portion of the (a) or (b).

In the present description, the lamellar titanosilicate is a generic name for a titanosilicate having a lamellar structure such as a lamellar precursor of a crystalline titanosilicate and a titanosilicate having an expanded interlayer space in a crystalline titanosilicate. The lamellar structure can be confirmed by an electron microscope.

The lamellar precursor means a titanosilicate which is converted into a crystalline titanosilicate by treatment such as dehydrative condensation. The dehydrative condensation can be conducted by heating the precursor generally at the temperature of 250 to 800° C. The fact that a lamellar titanosilicate has a pore composed 12- or more-membered oxygen ring can be easily confirmed from the structure of the corresponding crystalline titanosilicate.

The titanosilicates described in the above 1 to 3 and silylated products thereof usually comprise a pore having a pore size of 0.6 nm to 1.0 nm. In the present invention, a pore size means the diameter of (a) the cross-section of the narrowest portion of a pore or (b) the pore opening. In the present description, the pore size is generally determined by analyzing X-ray diffraction pattern of the titanosilicates.

The mesoporous titanosilicate is a generic name for a titanosilicate having a regular mesopore. The regular mesopore means a structure in which mesopores are regularly and repeatedly arranged. The mesopore mean a pore having a pore size of 2 nm to 10 nm.

The silylated titanosilicates are obtained by treating the titanosilicates described in the above 1 to 4 with a silylating agent. Examples of the silylating agent include 1,1,1,3,3,3 -hexamethyldisilazane and trimethylchlorosilane (for example, one described in EP 1488853A1).

Examples of the titanosilicate in the present invention include preferably Ti-MWW, a Ti-MWW precursor and a silylated Ti-MWW, and more preferably a Ti-MWW precursor.

The titanosilicate in the present invention may be used after activation by treatment with a hydrogen peroxide solution. The concentration of the hydrogen peroxide solution is usually in the range of 0.0001% by mass to 50% by mass. The solvent for the hydrogen peroxide solution is not particularly limited, but water or a solvent used in the propylene oxide synthesis reaction is convenient and preferable from the industrial viewpoint.

In the present invention, the mass ratio of a noble metal catalyst to the titanosilicate (the mass of a noble metal catalyst/the mass of the titanosilicate) is preferably 0.01 to 100% by mass and more preferably 0.1 to 100% by mass.

In the production method of the present invention, propylene oxide is produced in a mixed solvent of water and a nitrile compound. Examples of the nitrile compound include a linear or branched saturated aliphatic nitrile compound or aromatic nitrile compound. Specific examples of the nitrile compound include a C2-C4 alkylnitrile such as acetonitrile, propionitrile, isobutylonitrile and butylonitrile and a C6-C10 benzonitrile, and acetonitrile is preferred. Generally, the ratio by mass of water to the nitrile compound (water: the nitrile compound) is usually 90:10 to 0.01:99.99, preferably 50:50 to 0.1:99.9 and more preferably 40:60 to 5:95.

In the present invention, propylene oxide is produced from hydrogen, oxygen and propylene. Examples of the oxygen include a molecular oxygen such as oxygen gas. The oxygen gas may be an oxygen gas produced by an inexpensive pressure swing adsorption, and a high-purity oxygen gas produced by cryogenic separation can also be used if necessary. As the oxygen, air may be used. As the hydrogen, a hydrogen gas is generally used. The oxygen and the hydrogen can be diluted with an inert gas. Examples of the inert gas include nitrogen, argon, carbon dioxide, methane, ethane and propane. Although there is no particular limitation on the flow rate of the oxygen gas and the hydrogen gas and the concentration of the inert gas, they can be set according to other conditions such as the reaction scale.

The partial pressure ratio between oxygen and hydrogen is in the range of usually 1:50 to 50:1. The preferred partial pressure ratio between oxygen and hydrogen is in the range of 1:5 to 5:1. In any of the ranges, the reaction is preferably carried out outside the explosion range from the viewpoint of safety.

In the above production, the amount of propylene is not particularly limited, but the molar ratio of propylene to oxygen (propylene:oxygen) is in the range of preferably 1:5 to 5:1. If propylene oxide is produced in a continuous manner, propylene preferably has a concentration of 0.1 g/L to 1000 g/L based on the mixed solvent.

Examples of means for the reaction according to the present invention include a fixed-bed flow reactor and a slurry complete mixing flow reactor.

The reaction temperature in the present reaction is usually 0° C. to 150° C. and preferably 40° C. to 90° C. The reaction pressure is not particularly limited, but is usually 0.1 MPa to 20 MPa and preferably 1 MPa to 10 MPa in gauge pressure. After reaction, the liquid phase or the gas phase taken out from the reactor can be separated by distillation to obtain a target product.

In the present invention, an additive such as a polycyclic compound and a quinoid compound, which suppresses the production of propane obtained as a by-product, can be allowed to coexist. If the additive is allowed to coexist in the reaction system, the selectivity of propylene oxide based on hydrogen can be further increased. Specific examples of the additive include a polycyclic compound such as anthracene, tetracene, 9-methylanthracene, naphthalene, tetracene and diphenylether (for example, one described in JP 2009-23998 A) and a quinoid compound such as anthraquinone, 9,10-phenanthraquinone, benzoquinone and 2-ethylanthraquinone (for example, one described in JP 2008-106030 A). Among the additives, examples of a preferred additive include a condensed polycyclic aromatic compound such as anthracene, tetracene, 9-methylanthracene, naphthalene, tetracene, anthraquinone, 9,10-phenanthraquinone and 2-ethylanthraquinone. Examples of a more preferred additive include anthraquinone.

The amount of the additive is in the range of usually 0.001 mmol/kg to 500 mmol/kg and preferably 0.01 mmol/kg to 50 mmol/kg, per kg of the mixed solvent.

In the present invention, a salt composed of ammonium, an alkyl ammonium or alkyl-aryl ammonium (hereinafter, referred to as an ammonium salt) may be added to the mixed solvent. The use efficiency of hydrogen can be increased by the addition of the ammonium salt. Examples of ammonium salt include an ammonium sulfate and a hydrosulfate, and further, as ammonium salt, there may be added a salt of an inorganic acid such as a hydrogen carbonate salt, a phosphate salt, a hydrogenphosphate salt, a dihydrogenphosphate salt, a hydrogen pyrophosphate salt, a pyrophosphate salt, a halide salt and a nitrate salt, and a salt of an organic acid (for example, carboxylic acid) such as an acetate salt. Examples of a preferred additive include diammonium hydrogen phosphate. The amount added of the ammonium salt is usually 0.001 mmol/kg to 100 mmol/kg, per kg of the mixed solvent.

### EXAMPLES

Hereinafter, the present invention will be described with reference to Examples, but the present invention is not limited to these Examples.

### Reference Example 1

### Preparation of Titanosilicate Having MWW Precursor Structure

A gel was prepared by stirring 899 g of piperidine, 2402 g of purified water, 112 g of tetra-n-butylorthotitanate (TBOT), 565 g of boric acid and 410 g of fumed silica (cab-o-sil M7D) at room temperature in an air atmosphere in an autoclave. The resulting gel was aged for 1.5 hours and then the autoclave was sealed. Further, the gel was heated over 8 hours under stirring and maintained at 160° C. for 120 hours, thereby performing hydrothermal synthesis to obtain a suspension.

The resulting suspension was filtered and then washed with water until the pH of the filtrate reached approximately 10. Thereafter, a filter cake was dried at 50° C. until no reduction in the mass, and 515 g of a solid (a) was obtained. After addition of 3750 mL of 2M nitric acid to the 75 g of solid (a), the mixture was refluxed for 20 hours.

Subsequently, the resulting mixture was filtered and then washed with water until the pH of the filtrate became nearly neutral, followed by dried in vacuum at 150° C. until no reduction in the mass, and 61 g of a white powder (a) was obtained. The X-ray diffraction pattern and ultraviolet visible absorption spectra of the white powder (a) were measured, and it was confirmed that the white powder (a) is a Ti-MWW precursor by comparing the X-ray diffraction pattern in FIG. 1 of EP1731515A1, that is, has a pore composed 12-membered oxygen ring (hereinafter, referred to as “Ti-MWW precursor (a)”).

After calcining 60 g of the resulting white powder (a) at 530° C. for 6 hours, 54 g of powder (Ti-MWW) was obtained. The fact that the resulting powder has MWW structure by comparing the X-ray diffraction pattern in FIG. 2 of EP1731515A1, that is, has a pore composed 12-membered oxygen ring, was confirmed by measuring the X-ray diffraction pattern. In addition, the ultraviolet visible absorption spectra of the resulting powder were measured, and the results showed that the resulting powder was a titanosilicate. Further, the same operation as above was carried out twice to obtain a total of 162 g of Ti-MWW.

A gel was prepared by stirring 300 g of piperidine, 600 g of purified water and 135 g of the Ti-MWW obtained as described above at room temperature in an air atmosphere in an autoclave. The resulting gel was aged for 1.5 hours and then the autoclave was sealed. Further, the gel was heated over 4 hours under stirring and maintained at 160° C. for 24 hours, thereby performing hydrothermal synthesis to obtain a suspension.

The resulting suspension was filtered and then washed with water until the pH of the filtrate reached approximately 9. Thereafter, the resulting solid was dried at 150° C. in vacuum until no reduction in the mass was observed, to provide 141 g of a white powder (b). The X-ray diffraction pattern of the white powder (b) was measured, and it was confirmed that the white powder (b) exhibits the same X-ray diffraction pattern as the MWW precursor structure by comparing the X-ray diffraction pattern in FIG. 1 of EP1731515A1, that is, has a pore composed 12-membered oxygen ring. In addition, the ultraviolet visible absorption spectra of the white powder (b) were measured, and the results showed that the white powder (b) was a titanosilicate (hereinafter, referred to as “Ti-MWW precursor (b)”). Further, ICP emission analysis showed that the titanium content was 1.61% by mass.

In addition, the resulting Ti-MWW precursor and Ti-MWW are each stirred in 80 g of a mixed solution of water/acetonitrile=20/80 (mass ratio) containing 0.1% by mass of hydrogen peroxide for one hour, and the resulting mixture was filtered and washed with 80 g of water. The resulting solids were used in Examples.

### Reference Example 2

### Preparation of Pd/Activated Carbon (AC) Catalyst

Into a 1-L pear-shaped flask were added 300 mL of water and 6 g of activated carbon (produced by Wako Pure Chemical Industries Ltd.) which was preliminarily washed with 2 L of water, and the mixture was stirred at room temperature in air. To the resulting suspension was slowly dropwise added 100 mL of an aqueous solution containing 0.60 mmol of a Pd colloid (produced by JGC Catalysts and Chemicals Ltd.) at room temperature in air. After completion of the dropwise addition, the suspension was further stirred at room temperature in air for 8 hours. After completion of the stirring, water was removed with a rotary evaporator, and the residue was dried in vacuum at 80° C. for 6 hours and further calcined at 300° C. for 6 hours in a nitrogen atmosphere to obtain a Pd/AC catalyst. ICP emission analysis showed that the Pd content was 0.95% by mass.

### Example 1

An autoclave having a capacity of 0.3 L was used as a reactor. To the reactor were fed a raw material gas having a volume ratio of propylene/oxygen/hydrogen/nitrogen of 8/4.2/4.4/83.4 at a rate of 20 L/hr and a solution of water/acetonitrile=20/80 (mass ratio) containing 0.7 mmol/kg of anthraquinone and 9 μmol/kg of diphenyl sulfide at a rate of 108 mL/hr, and a solution containing a reaction product and a gas were taken out through a filter from the reaction mixture in the reactor, thereby performing a continuous reaction under the conditions of a temperature of 60° C., a pressure of 0.8 MPa (gauge pressure) and a residence time of 90 minutes. During this period, the reaction was carried out by maintaining the amount of the mixed solvent at 133 g, the amount of the titanosilicate (Ti-MWW precursor (b)) at 0.6 g and the amount of Pd/AC at 0.06 g in the reactor. 5 hours later from the beginning of the reaction, the liquid and gas phases were taken out from the reactor and analyzed by gas chromatography. A production activity of the propylene oxide per unit mass of titanosilicate was 15.7 mmol-PO/g-titanosilicate·h, the selectivity based on propylene was 97% and the selectivity based on hydrogen (molar amount of formed propylene oxide/molar amount of consumed hydrogen) was 84%.

### Example 2

The reaction was carried out in the same manner as in Example 1 except for using a solution of water/acetonitrile=20/80 (mass ratio) containing 0.7 mmol/kg of anthraquinone and 450 μmol/kg of diphenyl sulfone instead of a solution of water/acetonitrile=20/80 (mass ratio) containing 0.7 mmol/kg of anthraquinone and 9 μmol/kg of diphenyl sulfide.

5 hours later from the beginning of the reaction, the liquid and gas phases were taken out from the reactor and analyzed by gas chromatography. A production activity of the propylene oxide per unit mass of titanosilicate was 18.5 mmol-PO/g-titanosilicate·h, the selectivity based on propylene was 97% and the selectivity based on hydrogen was 81%.

### Example 3

The reaction was carried out in the same manner as in Example 1 except for using a solution of water/acetonitrile=20/80 (mass ratio) containing 0.7 mmol/kg of anthraquinone and 9 μmol/kg of methyl phenyl sulfide instead of a solution of water/acetonitrile=20/80 (mass ratio) containing 0.7 mmol/kg of anthraquinone and 9 μmol/kg of diphenylsulfide.

5 hours later from the beginning of the reaction, the liquid and gas phases were taken out from the reactor and analyzed by gas chromatography. A production activity of the propylene oxide per unit mass of titanosilicate was 17.1 mmol-PO/g-titanosilicate·h, the selectivity based on propylene was 96% and the selectivity based on hydrogen was 86%.

### Example 4

The reaction was carried out in the same manner as in Example 1 except for using a solution of water/acetonitrile=20/80 (mass ratio) containing 0.7 mmol/kg of anthraquinone and 9 μmol/kg of dibutyl sulfide instead of a solution of water/acetonitrile=20/80 (mass ratio) containing 0.7 mmol/kg of anthraquinone and 9 μmol/kg of diphenyl sulfide.

5 hours later from the beginning of the reaction, the liquid and gas phases were taken out from the reactor and analyzed by gas chromatography. A production activity of the propylene oxide per unit mass of titanosilicate was 18.2 mmol-PO/g-titanosilicate·h, the selectivity based on propylene was 97% and the selectivity based on hydrogen was 78%.

Example 5

The reaction was carried out in the same manner as in Example 1 except for using a solution of water/acetonitrile=20/80 (mass ratio) containing 0.7 mmol/kg of anthraquinone and 450 μmol/kg of dimethyl sulfide instead of a solution of water/acetonitrile=20/80 (mass ratio) containing 0.7 mmol/kg of anthraquinone and 9 μmol/kg of diphenyl sulfide.

5 hours later from the beginning of the reaction, the liquid and gas phases were taken out from the reactor and analyzed by gas chromatography. A production activity of the propylene oxide per unit mass of titanosilicate was 18.7 mmol-PO/g-titanosilicate·h, the selectivity based on propylene was 98% and the selectivity based on hydrogen was 79%.

Example 6

The reaction was carried out in the same manner as in Example 1 except for using a solution of water/acetonitrile=20/80 (mass ratio) containing 0.7 mmol/kg of anthraquinone and 39 μmol/kg of 2,2′-thiodiethanol instead of a solution of water/acetonitrile=20/80 (mass ratio) containing 0.7 mmol/kg of anthraquinone and 9 μmol/kg of diphenylsulfide.

5 hours later from the beginning of the reaction, the liquid and gas phases were taken out from the reactor and analyzed by gas chromatography. A production activity of the propylene oxide per unit mass of titanosilicate was 14.9 mmol-PO/g-titanosilicate·h, the selectivity based on propylene was 94% and the selectivity based on hydrogen was 79%.

### Example 7

The reaction was carried out in the same manner as in Example 1 except for using a solution of water/acetonitrile=20/80 (mass ratio) containing 700 μmol/kg of diphenyl sulfone instead of a solution of water/acetonitrile=20/80 (mass ratio) containing 0.7 mmol/kg of anthraquinone and 9 μmol/kg of diphenylsulfide. 5 hours later from the beginning of the reaction, the liquid and gas phases were taken out from the reactor and analyzed by gas chromatography. A production activity of the propylene oxide per unit mass of titanosilicate was 15.5 mmol-PO/g-titanosilicate·h, the selectivity based on propylene was 69% and the selectivity based on hydrogen was 39%.

### Example 8

The reaction was carried out in the same manner as in Example 1 except for using a solution of water/acetonitrile=20/80 (mass ratio) containing 700 μmol/kg of sulfolane instead of a solution of water/acetonitrile=20/80 (mass ratio) containing 0.7 mmol/kg of anthraquinone and 9 μmol/kg of diphenyl sulfide. 5 hours later from the beginning of the reaction, the liquid and gas phases were taken out from the reactor and analyzed by gas chromatography. A production activity of the propylene oxide per unit mass of titanosilicate was 9.6 mmol-PO/g-titanosilicate·h, the selectivity based on propylene was 47% and the selectivity based on hydrogen was 23%.

### Example 9

An autoclave having a capacity of 0.3 L was used as a reactor. To the reactor were fed a raw material gas having a volume ratio of oxygen/hydrogen/nitrogen of 3.3/3.6/93.1 at a rate of 281 L/hr, a solution of water/acetonitrile=30/70 (mass ratio) containing 0.7 mmol/kg of anthraquinone, 3.0 mmol/kg of diammonium hydrogen phosphate and 20 μmol/kg of diphenylsulfide at a rate of 90 g/hr and propylene at a rate of 36 g/hr, and a solution containing a reaction product and a gas were taken out through a filter from the reaction mixture in the reactor, thereby performing a continuous reaction under the conditions of a temperature of 50° C., a pressure of 4.0 MPa (gauge pressure) and a residence time of 60 minutes. The reaction was carried out by maintaining the amount of the mixed solvent at 133 g, the amount of the titanosilicate (Ti-MWW precursor (b)) at 2.28 g and the amount of Pd/AC at 1.05 g in the reactor. 6 hours later from the beginning of the reaction, the liquid and gas phases were taken out from the reactor and analyzed by gas chromatography. A production activity of the propylene oxide per unit mass of titanosilicate was 82.0 mmol-PO/g-titanosilicate·h, the selectivity based on propylene was 91% and the selectivity based on hydrogen was 75%.

### Reference Example 3

### Preparation of Ph₂S-Treated Pd/AC Catalyst

The activated carbon used was prepared by drying 20 g of activated carbon (produced by Wako Pure Chemical Industries Ltd.), which was washed with 10 L of hot water (100° C.), at 150° C. under a nitrogen flow for 6 hours. Into a 1-L pear-shaped flask were added 6 g of the activated carbon and 1 L of water, and the mixture was stirred at room temperature in air. To the resulting suspension was slowly dropwise added 100 mL of an aqueous solution containing 0.60 mmol of a Pd colloid (produced by JGC Catalysts and Chemicals Ltd.) at room temperature in air. After completion of the dropwise addition, the suspension was further stirred at room temperature in air for 8 hours. After completion of the stirring, water was removed with a rotary evaporator, and the residue was dried in vacuum at 80° C. for 6 hours to obtain a black powder.

The black powder obtained as described above was washed with 2 L of water and 3 L of hot water (100° C.) in this order, followed by drying at 150° C. under a nitrogen flow for 6 hours to obtain a Pd/AC catalyst. ICP emission analysis showed that the S (sulfur) content was 0.041% by mass.

Into a 10-mL two-necked pear-shaped flask were added 0.6 g of the Pd/AC obtained by the above method and 8 mL of a methanol solution containing 0.021 g of diphenyl sulfide and the mixture was stirred at room temperature in air for 5 days. The resulting suspension was filtered and then washed with methanol and diethylether, followed by dried in vacuum at 50° C. for 2 hours to obtain a Ph₂S-treated Pd/AC catalyst. ICP emission analysis showed that the Pd content and the S (sulfur) content were 1.06% by mass and 0.067% by mass, respectively.

### Example 10

An autoclave having a capacity of 0.3 L was used as a reactor. To the reactor were fed a raw material gas consisting of a volume ratio of propylene/oxygen/hydrogen/nitrogen of 8/4.2/4.4/83.4 at a rate of 20 L/hr and a solution of water/acetonitrile=20/80 (mass ratio) containing 0.7 mmol/kg of anthraquinone at a rate of 108 mL/hr, and a solution containing a reaction product and a gas were taken out through a filter from the reaction mixture in the reactor, thereby performing a continuous reaction under the conditions of a temperature of 60° C., a pressure of 0.8 MPa (gauge pressure) and a residence time of 90 minutes. The reaction was carried out by maintaining the amount of the mixed solvent at 133 g, the amount of the titanosilicate at 0.6 g of Ti-MWW precursor (b) and the amount of the Ph₂S-treated Pd/AC catalyst prepared by the method of Reference Example 3 at 0.06 g in the reactor. 5 hours later from the beginning of the reaction, the liquid and gas phases were taken out from the reactor and analyzed by gas chromatography. A production activity of the propylene oxide per unit mass of titanosilicate was 15.9 mmol-PO/g-titanosilicate·h, the selectivity based on propylene was 98% and the selectivity based on hydrogen (molar amount of formed propylene oxide/molar amount of consumed hydrogen) was 88%.

### Comparative Example 1

The reaction was carried out in the same manner as in Example 1 except for using a solution of water/acetonitrile=20/80 (mass ratio) containing 0.7 mmol/kg of anthraquinone instead of a solution of water/acetonitrile=20/80 containing 0.7 mmol/kg of anthraquinone and 9 μmol/kg of diphenylsulfide.

5 hours later from the beginning of the reaction, the liquid and gas phases were taken out from the reactor and analyzed by gas chromatography. A production activity of the propylene oxide per unit mass of titanosilicate was 16.8 mmol-PO/g-titanosilicate·h, the selectivity based on propylene was 96% and the selectivity based on hydrogen was 70%.

### Comparative Example 2

The reaction was carried out in the same manner as in Example 7 except for using a solution of water/acetonitrile=20/80 (mass ratio) containing no additives instead of a solution of water/acetonitrile=20/80 containing 700 μmol/kg of diphenylsulfone.

5 hours later from the beginning of the reaction, the liquid and gas phases were taken out from the reactor and analyzed by gas chromatography. A production activity of the propylene oxide per unit mass of titanosilicate was 3.6 mmol-PO/g-titanosilicate·h, the selectivity based on propylene was 22% and the selectivity based on hydrogen was 11%.

### Comparative Example 3

The reaction was carried out in the same manner as in Example 9 except for using a solution of water/acetonitrile=30/70 (mass ratio) containing 0.7 mmol/kg of anthraquinone and 3.0 mol/kg of diammonium hydrogen phosphate instead of a solution of water/acetonitrile=30/70 containing 0.7 mmol/kg of anthraquinone, 3.0 mmol/kg of diammonium hydrogen phosphate and 20 μmol/kg of diphenylsulfide.

5 hours later from the beginning of the reaction, the liquid and gas phases were taken out from the reactor and analyzed by gas chromatography. A production activity of the propylene oxide per unit mass of titanosilicate was 74.1 mmol-PO/g-titanosilicate·h, the selectivity based on propylene was 92% and the selectivity based on hydrogen was 61%.

### Comparative Example 4

The reaction was carried out in the same manner as in Example 10 except for using Pd/AC which was not treated with diphenylsulfide instead of the Ph₂S-treated Pd/AC catalyst.

5 hours later from the beginning of the reaction, the liquid and gas phases were taken out from the reactor and analyzed by gas chromatography. A production activity of the propylene oxide per unit mass of titanosilicate was 16.9 mmol-PO/g-titanosilicate·h, the selectivity based on propylene was 97% and the selectivity based on hydrogen was 70%.

### INDUSTRIAL APPLICABILITY

The present invention may be applicable to a reaction for producing propylene oxide from hydrogen, oxygen and propylene.

1. A method for producing propylene oxide, comprising the step of producing propylene oxide from hydrogen, oxygen and propylene in the presence of a noble metal catalyst, a titanosilicate and an organic sulfur compound in a mixed solvent of water and a nitrile compound, wherein the titanosilicate has a pore composed 12- or more-membered oxygen ring.

2. The method according to claim 1, wherein the organic sulfur compound is selected from the group consisting of a sulfide compound, a sulfoxide compound and a sulfone compound.

3. The method according to claim 1, wherein the organic sulfur compound is selected from the group consisting of a sulfide compound and a sulfone compound.

4. The method according to claim 2, wherein the sulfide compound is a dialkyl sulfide, an alkyl aryl sulfide or a diaryl sulfide.

5. The method according to claim 2, wherein the sulfoxide compound is a dialkyl sulfoxide, an alkyl aryl sulfoxide or a diaryl sulfoxide.

6. The method according to claim 2, wherein the sulfone compound is a dialkyl sulfone, an alkyl aryl sulfone or a diaryl sulfone.

7. The method according to claim 1, wherein the organic sulfur compound is selected from the group consisting of a C2-C12 sulfide compound and a C2-C12 sulfone compound.

8. The method according to claim 1, wherein the organic sulfur compound is dissolved in the mixed solvent.

9. The method according to claim 1, wherein the nitrile compound is acetonitrile.

10. The method according to claim 1, wherein the noble metal catalyst is a noble metal selected from the group consisting of palladium, platinum, ruthenium, rhodium, iridium, osmium and gold, or an alloy comprising two or more kinds of the noble metals.

11. The method according to claim 10, wherein the noble metal catalyst is palladium, an alloy comprising palladium and the noble metal, or a mixture of the palladium and the alloy.

12. The method according to claim 1, wherein the noble metal catalyst is supported on a carrier.

13. The method according to claim 12, wherein the carrier is activated carbon.

14. The method according to claim 1, wherein the organic sulfur compound and the noble metal catalyst are supported on a carrier.

15. The method according to claim 1, wherein the titanosilicate is Ti-MWW, a Ti-MWW precursor or silylated Ti-MWW.

16. The method according to claim 1, wherein anthraquinone is contained in the mixed solvent.
